(19) United States
(12) Plant Patent Application Publication
(10) Pub. No.: US 2017/0367237 P1
(43) Pub. Date: Dec. 21, 2017

(54) Pelargonium plant named 'KLEPZ16424'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 15/731,359

(22) Filed: Jun. 1, 2017

(30) Foreign Application Priority Data
Jun. 16, 2016 (CH) 16-3070

(51) Int. Cl. A01H 5/02

(52) U.S. Cl. CPC A01H 5/0277
USPC PLT/327

(57) ABSTRACT

A new pelargonium plant particularly distinguished by large, salmon orange colored flowers with purple eyes and an upright growth habit, is disclosed.

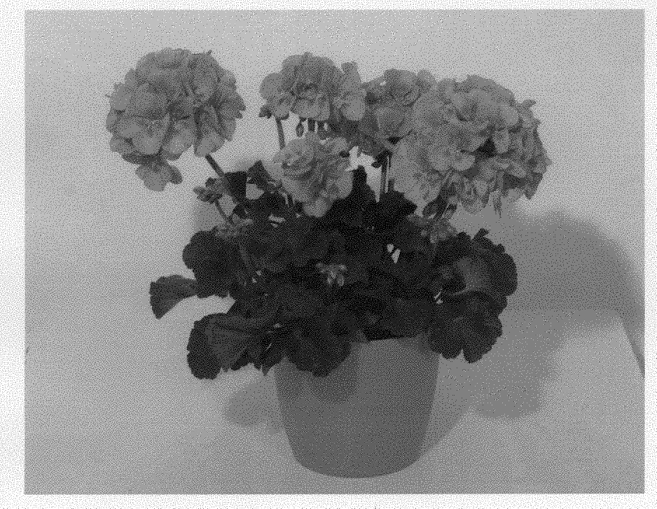

### CROSS REFERENCE TO RELATED APPLICATION

The present application claims priority to Switzerland Plant Variety Protection Application No. 16-3070, as filed on Jun. 16, 2016, the entire contents are herein incorporated by reference for all the application teaches and discloses.

Genus and species: Pelargonium zonale.

Variety denomination: ‘KLEPZ16424’.

### BACKGROUND OF THE NEW PLANT

The present invention comprises a new and distinct variety of pelargonium, botanically known as Pelargonium zonale and hereinafter referred to by the variety name ‘KLEPZ16424’. ‘KLEPZ16424’ originated from a cross-combination in September 2010 in Stuttgart, Germany between the female pelargonium variety ‘KLEPZ05129’, also known as SUNRISE Mandarin (U.S. Plant Pat. No. 18,621) and the proprietary male pelargonium variety SUNRISE Racing (unpatented).

Seeds from the cross-combination were obtained and sown and plants were grown for evaluation. A single plant was selected in June 2011 and subsequently named ‘KLEPZ16424’. In August 2011 ‘KLEPZ16424’ was first vegetatively propagated in Stuttgart, Germany by vegetative cuttings from branches. ‘KLEPZ16424’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. Large salmon-orange colored flowers with purple eyes; and

2. An upright growth habit.

### DESCRIPTION OF THE PHOTOGRAPH

This new pelargonium plant is illustrated by the accompanying photograph which shows an overall view of the plant. The photograph is of an approximately nine to ten-week-old plant grown in Stuttgart, Germany under greenhouse conditions in May 2015. The colors shown are as true as can be reasonably obtained by conventional photographic procedures.

### DESCRIPTION OF THE NEW VARIETY

The following detailed descriptions set forth the distinctive characteristics of ‘KLEPZ16424’. The data which define these characteristics were collected from asexual reproductions carried out in Stuttgart, Germany. Data was collected on plants grown three months after planting in Stuttgart, Germany under a glass covered greenhouse in April 2017. Color references are to The R.H.S. Colour Chart of The Royal Horticultural Society of London (R.H.S.), 5^{th} edition (2007).
Classification:
   Family.—Geraniaceae.
   Botanical.—Pelargonium zonale.
   Common.—Pelargonium.
   Designation.—‘KLEPZ16424’.
Parentage:
   Female parent.—The female pelargonium variety ‘KLEPZ05129’ (U.S. Plant Pat. No. 18,621).
   Male parent.—The proprietary male pelargonium variety SUNRISE Racing (unpatented).
Plant:
   Form.—Bushy and round.
   Habit.—Upright.
   Height.—16.0 cm.
   Spread.—25.0 cm.
   Natural flowering season.—Spring to autumn.
   Propagation type.—Vegetative cuttings.
   Time to produce a finished flowering plant.—4 months.
   Time to initiate and develop roots.—20 days.
   Root description.—White and fine.
Stems:
   Quantity of stems per plant.—4 to 5.
   Color.—RHS 138A.
   Surface texture.—Sparsely pubescent.
   Stem form.—Round.
   Diameter.—1.0 cm.
   Length.—8.0 cm.
   Internode length.—1.5 cm to 2.0 cm.
   Anthocyanin.—Absent.
Leaves:
   Arrangement.—Alternate.
   Shape.—Transverse.
   Apex.—Round.
   Base.—Open.
   Margin.—Crenate.
   Surface texture.—Upper surface: Sparsely pubescent Lower surface: Sparsely pubescent
   Length.—4.0 cm to 6.0 cm
   Width.—6.0 cm to 8.0 cm.
   Color.—Upper surface: RHS N137B Lower surface: RHS 137B.
   Petiole.—Length: 4.0 cm to 6.0 cm Diameter: 0.3 cm Color: RHS 138A Strength: Medium.
   Venation pattern.—Palmate.
   Venation color.—Upper surface: RHS 137B Lower surface: RHS 138A.
Inflorescence:
   Lastingness of flowers on the plant.—6 to 8 days.
   Umbels.—Quantity of umbels per plant: 3 to 4 Length: 8.0 cm Diameter: 11.0 cm.
   Individual florets of the umbel.—Quantity of florets per umbel: Greater than 25 Depth: 2.0 cm Diameter: 4.5 cm.
   Fragrance.—Absent.
Flower buds:
   Cluster shape.—Round.
   Cluster size.—Length: 2.5 cm Width: 3.0 cm.
   Surface texture.—Moderately pubescent. Bud: Height: 0.8 cm Width: 0.6 cm Shape: Elliptic Apex: Acute Base: Obtuse Margin: Entire Color: RHS 138B.
Petals:
   Quantity per flower.—5.
   Shape.—Spatulate.
   Apex.—Round.
   Base.—Obtuse.
   Margin.—Entire.
   Length.—3.0 cm.
   Width.—2.5 cm.
   Surface texture.—Upper surface: Smooth Lower surface: Smooth
   Color of lobes.—Upper lobes: Upper surface: RHS 43C with eye of RHS N57A Lower surface: RHS 43D Lower lobes: Upper surface: RHS 43C with eye of RHS N57A Lower surface: RHS 43D.
Petaloids:
   Quantity per flower.—0 to 2.
   Shape.—Moderate.
   Apex.—Acute.
   Base.—Obtuse.
   Margin.—Entire.
   Length.—1.0 cm.
   Width.—0.2 cm to 0.8 cm.
   Texture (both surfaces).—Smooth.
   Color.—Upper surface: RHS 43D Lower surface: RHS 39D.
Sepals:
   Number per flower.—5.
   Shape.—Elliptic.
   Apex.—Acute.
   Base.—Obtuse.
   Margin.—Entire.
   Surface texture.—Upper surface: Smooth Lower surface: Moderately pubescent.
   Color.—Upper surface: RHS 138B Lower surface: RHS 138B
   Length.—1.5 cm.
   Width.—0.4 cm.
Pedicel:
   Length.—2.0 cm.
   Diameter.—0.15 cm.
   Color.—RHS 138B.
   Surface texture.—Sparsely pubescent.
   Strength.—Medium.
Peduncle:
   Length.—12.0 cm.
   Diameter.—0.7 cm.
   Color.—RHS 137B.
   Surface texture.—Moderately pubescent.
   Strength.—Medium to strong.
Reproductive Organs:
   Stamens.—Quantity of stamens per flower: 5 Anther length: 0.2 cm Filament length: 0.8 cm Filament color: RHS 69D Pollen amount: Moderate Pollen color: RHS 31B.
   Pistils.—Number: 1 Stigma color: RHS 38C Style length: 0.3 cm Style color: RHS 69D Ovary arrangement: Central Ovary color: RHS 138C Ovary diameter: 0.3 cm Ovary length: 0.1 cm.
Fruit and seed set: None observed.
Disease and pest/insect resistance: None observed.
Comparison with parental lines and known variety: ‘KLEPZ16424’ is distinguished from its parents as described in Table 1:

TABLE 1
Comparison with Parental Lines

| | | Female parent | Male Parent |
|---|---|---|---|
| Characteristic | ‘KLEPZ16424’ | ‘KLEPZ05129’ | SUNRISE Racing |
| Growth | Medium | Compact to Medium | Strong |
| Flower color | Salmon orange | Orange | Salmon cerise |
| | with purple eye | | |
| Leaf zonation | Absent to very | Weak | Absent |
| | weak | | |

‘KLEPZ16424’ can be compared to the commercial variety ‘Orangepen’ (patent status unknown). Differences between the two varieties are described in Table 2:

TABLE 2
Comparison with Similar Variety

| | | Commercial line |
|---|---|---|
| Characteristic | ‘KLEPZ16424’ | ‘Orangepen’ |
| Flower color | Salmon orange with purple | Orange |
| | eye | |
| Flower size | Large | Medium |

We claim:

1. A new and distinct variety of pelargonium plant named ‘KLEPZ16424’ as illustrated and described herein.
